(19) United States
(12) Patent Application Publication
(10) Pub. No.: US 2022/0168246 A1
(43) Pub. Date: Jun. 2, 2022

(54) COMPOSITONS FOR THE TREATMENT OF A CONDITION CHARACTERIZED BY ANANDAMIDE DEFICIENCY AND USES THEREOF

(71) Applicant: BUZZELET DEVELOPMENT AND TECHNOLOGIES LTD., Or-Akiva (IL)

(72) Inventors: AHARON EYAL, JERUSALEM (IL); NOA RAZ, GIZO (IL)

(21) Appl. No.: 17/673,845

(22) Filed: Feb. 17, 2022

Related U.S. Application Data
(63) Continuation-in-part of application No. PCT/IB2020/057805, filed on Aug. 19, 2020.
(60) Provisional application No. 62/888,562, filed on Aug. 19, 2019.
(60) Provisional application No. 62/888,560, filed on Aug. 19, 2019.

(51) Int. Cl. A61K 31/16; A61K 31/05; A61K 31/352; A61K 31/01

(52) U.S. Cl. CPC A61K 31/16; A61K 31/01; A61K 31/352; A61K 31/05

(57) ABSTRACT

Pharmaceutical compositions having a carrier and a pharmaceutically effective amount of at least one modulator of an activity which is responsive to modulation by at least one selected from the group consisting of anadamide, N-Oleoylethanolamine (OEA), N-palmitoylethanolamind (PEA) and seretonine and is responsive to modulation by 2-arachidonoylglycerol (2-AG) to an extent that is no greater than that provided by at least one of anadamide, OEA, PEA and seretonine at the same molar dose, wherein the composition is devoid of a sole modulator selected from the group consisting of cannabidiol (CBD), anadamide N-Oleoylethanolamine (OEA), N-palmitoylethanolamind (PEA) and seretonine, for use in treating a condition characterized by anandamide deficiency or a symptom thereof, pharmaceutical preparations and kits including the composition, and methods of use thereof.

### FIELD OF THE INVENTION

The field of art to which this invention generally pertains is pharmaceutical compositions, and more specifically to pharmaceutical compositions and uses thereof for treating a condition characterized by anandamide deficiency or a symptom thereof.

### BACKGROUND OF THE INVENTION

Anandamide (N-arachidonoylethanolamine or AEA) —C₂₂H₃₇NO₂— is an endocannabinoid which acts as a neurotransmitter and has a structure very similar to that of tetrahydrocannabinol (THC), the active constituent of cannabis. Anandamide affects the endocannabinoid system, which comprises the endocannabinoids, their producing enzymes, their degrading enzymes and their receptors. Anandamide is a component of the endocannabinoid system. The endocannabinoid system comprises endocannabiniods, their producing enzymes, their degrading enzymes and their receptors. Endocannabinoid receptors include G protein-coupled receptors, e.g. CB1, CB2, GPCR119, GPCR55, transient receptor potential (TRP) channel, peroxisome proliferator-activated receptor (PPAR) and others. Modulating of these receptors by anadamide or by other endocannabionids affects the activity of the endocannabinoid system. Endocannabinoids and their receptors are found throughout the body: in the brain, lungs, digestive system, connective tissues, hormone releasing glands, skin/hair, bone, the immune system, and the reproductive organs. The effects of ananadamide can occur in either the central or peripheral nervous system, mediated primarily by CB1 cannabinoid receptors in the central nervous system, and CB2 cannabinoid receptors in the periphery.

Palmitoylethanolamide (PEA) and oleoylethanolamide (OEA)N-acylethanolamine are N-acylethanolamine analogs of AEA. Synthesis of AEA, OEA and PEA is catalyzed by N-acylphospatidylethanolamine-specific phospholipase-D (NAPE-PLD) and their degradation is catalyzed by fatty acid amide hydrolase (FAAH).

2-arachidonoylglycerol (2-AG) is a monoacylglycerol endocannabionoid. Synthesis of 2-AG is catalyzed by diacylglycerol lipase (DAGL) and its degradation is catalyzed by monoacylglycerol lipase (MAGL).

### SUMMARY OF THE INVENTION

According to an aspect of some embodiments of the present invention, there is provided a pharmaceutical composition comprising a carrier and a pharmaceutically effective amount of at least two modulators of an activity which activity is responsive to modulation by at least one selected from the group consisting of anadamide, N-Oleoylethanolamine (OEA), N-palmitoylethanolamind (PEA) and seretonine and which activity is responsive to modulation by 2-arachidonoylglycerol (2-AG) to an extent that is no greater than that provided by at least one of anadamide, OEA, PEA and seretonine at the same molar dose.

According to some embodiments, at least one modulator is selected from the group consisting of transient receptor potential (TRP) channel modulators, TRPV1 channel modulators, peroxisome proliferator-activated receptor (PPAR) modulators, serotonin (5-HT) receptor modulators, glycine receptor (GlyR) modulators, G protein-coupled receptor modulators, G protein-coupled receptor 119 (GPR119) receptor modulators, cannabinoid receptor type 1 (CB1) modulators and combinations thereof.

According to some embodiments, at least one modulator comprises at least one selected from a group comprising neutral cannabidiol (CBD), cannabiodiolic acid (CBDA) neutral tetrahydrocannabinol (THC), tetrahydrocannbinolic acid (THCA), cannabivarin (CBDV), of N-palmitoylethanolamide (PEA), N-oleoylethanolamide (OEA), terpenes and combination thereof.

According to some embodiments, at least one modulator is selected from the group consisting of Capsaicin, capsaicinoid, capsinoid, Capsiate, Dihydrocapsiate, Nordihydrocapsiate, Piperine, Eugenol, Resiniferatoxin, Gingerol, Shogaol, Zingerone, Paradol, Grifolin, Neogrifolin, Albaconol, Scutigeral, Cochinchinenin, Loureirin, Allyl isothiocyanate, Cinnamaldehyde, Allicin, Diallyl disulphide, Ligustilide, Dehydroligustilide, Alpha-spinasterol, Guaiacol, Methyl salicylate, Vanillin, Methyl syringate, Resveratrol, Pinosylvin methyl ether, Salidroside, Bisandrographolide, Ginsenoside, Triptolide, Evodiamine, Nicotine, Yohimbine, Hydroxy-alphasanshool, Artepillin, Thapsigargin, Ricinoleic acid, Incensole acetate, Hyperforin, Vanillotoxins, Quinine, Waixenicin, Citral, Camphor, Carvacrol, Thymol, Menthol, cineole, Perillaldehyde, Perillaketone, Polygodial, Drimanial, Isovelleral, Cinnamodial, Cinnamosmolide, Cinnamolide, Warburganal, Scalaradial, Aframodial, Ancistrodial, Merulidial, Drimenol, Umbellulone, Cineole, Carveol, 6-tert-butyl-m-cresol, Dihydrocarveol, Borneol, Perillaldehyde, Perillaketone, oleoylethanolamide, N-oleoyldopamine, 3-methyl-N-oleoyldopamine, oleamide, 1-monoacylglycerol, 2-monoacylglycerol, 1-Arachidonoylglycerol, 2-arachidonoylglycerol, miogadial, miogatrial, polygodial, terpenes with an alpha,beta-unsaturated 1,4-dialdehyde moiety, sanshool, evodiamine, acesulfame-K, cyclamate, CuSO4, ZnSO4, FeSO4, arvanil, N-arachidonoyl-dopamine, flufenamic acid dopamide, dopamine amide of fenamic acid, 4-hydroxynonenal, 1-[2-(1-adamantyl)ethyl]-1-pentyl-3-[3-(4-pyridyl)propyl]urea, Alosetron, Dolasetron, Clozapine, Quetiapine, Mianserin, Piboserod, L-lysine, boldine, oleoyl LPC, N-oleoyldopamine, olvanil, N-palmitoylethanolamide (PEA) and N-oleoylethanolamide (OEA) and combinations thereof.

According to some embodiments, the pharmaceutical composition further comprises at least one terpene selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

According to an aspect of some embodiments of the present invention, there is provided a pharmaceutical preparation comprising the pharmaceutical composition as disclosed herein.

According to some embodiments, the preparation comprises between 0.1 and 100 milligram cannabidiol.

According to an aspect of some embodiments of the present invention, there is provided a kit comprising a first and a second preparation each comprising the composition as disclosed herein, wherein each of the first and the second preparations further comprises both cannabidiol and tetrahydrocannabinol and wherein a cannabidiol to tetrahydrocannabinol weight per weight ratio in the first preparation is greater than a cannabidiol to tetrahydrocannabinol weight per weight ratio in the second preparation.

According to an aspect of some embodiments of the present invention, there is provided a kit comprising a first and a second preparation each comprising the composition according to claim 1, wherein each of the first and the second preparations comprises at least one terpene, and wherein the terpene content of the first preparation differs from the terpene content of the second preparation.

According to an aspect of some embodiments of the present invention, there is provided a method for treating a condition characterized by anandamide deficiency or a symptom thereof in a subject in need thereof, comprising administering to the subject a pharmaceutical composition comprising a carrier and a pharmaceutically effective amount of at least one modulator of an activity which activity is responsive to modulation by at least one selected from the group consisting of anadamide, N-Oleoylethanolamine (OEA), N-palmitoylethanolamind (PEA) and seretonine and which activity is responsive to modulation by 2-arachidonoylglycerol (2-AG) to an extent that is no greater than that provided by at least one of anadamide, OEA, PEA and seretonine at the same molar dose, wherein the composition is devoid of a sole modulator selected from the group consisting of cannabidiol (CBD), anadamide N-Oleoylethanolamine (OEA), N-palmitoylethanolamind (PEA) and seretonine.

According to some embodiments, the at least one modulator is selected from the group consisting of transient receptor potential (TRP) channel modulators, peroxisome proliferator-activated receptor (PPAR) modulators, serotonin (5-HT) receptor modulators, glycine receptor (GlyR) modulators, G protein-coupled receptors modulators, G protein-coupled receptor 119 (GPR119) receptor modulators, cannabinoid receptor type 1 (CB1) modulators and combinations thereof.

According to some embodiments, the at least one modulator further comprises at least one selected from the group consisting of neutral cannabidiol (CBD), cannabiodiolic acid (CBDA), neutral tetrahydrocannabinol (THC), tetrahydrocannbinolic acid (THCA), cannabivarin (CBDV), of N-palmitoylethanolamide (PEA), N-oleoylethanolamide (OEA), terpenes and combination thereof.

According to some embodiments, the CBD is present in the composition at less than 50% of a total molar amount of the at least one modulator.

According to some embodiments, the condition involves no significant 2-AG deficiency.

According to some embodiments, the condition is selected from the group consisting of autistic spectrum disorders, migraine, fibromyalgia, irritable bowel diseases, diabetes, post-traumatic stress disorders, chronic stress, anxiety, stress-related psychiatric illness, hyperanalgesia, affective disorders, increased pain perception, reduced pain perception, headache, self-injury behavior, hyper-sensory processing, hypo-sensory processing, impaired sensory processing, hypersensitivity, hyposensitivity, impaired thermo-regulation, inflammation, digestive problems, nausea, vomiting, constipation, urinary-system related problems, impaired memory, impaired learning, seizures, anxiety, stress, fear, aversive emotion, aversive emotional memories, mood, depression, dermatitis, impaired social cognition, impaired social behavior, sleep problems, insomnia, an autism spectrum disorder and combinations thereof.

According to some embodiments, the at least one modulator is selected from the group consisting of Capsaicin, capsaicinoid, capsinoid, Capsiate, Dihydrocapsiate, Nordihydrocapsiate, Piperine, Eugenol, Resiniferatoxin, Gingerol, Shogaol, Zingerone, Paradol, Grifolin, Neogrifolin, Albaconol, Scutigeral, Cochinchinenin, Loureirin, Allyl isothiocyanate, Cinnamaldehyde, Allicin, Diallyl disulphide, Ligustilide, Dehydroligustilide, Alpha-spinasterol, Guaiacol, Methyl salicylate, Vanillin, Methyl syringate, Resveratrol, Pinosylvin methyl ether, Salidroside, Bisandrographolide, Ginsenoside, Triptolide, Evodiamine, Nicotine, Yohimbine, Hydroxy-alphasanshool, Artepillin, Thapsigargin, Ricinoleic acid, Incensole acetate, Hyperforin, Vanillotoxins, Quinine, Waixenicin, Citral, Camphor, Carvacrol, Thymol, Menthol, cineole, Perillaldehyde, Perillaketone, Polygodial, Drimanial, Isovelleral, Cinnamodial, Cinnamosmolide, Cinnamolide, Warburganal, Scalaradial, Aframodial, Ancistrodial, Merulidial, Drimenol, Umbellulone, Cineole, Carveol, 6-tert-butyl-m-cresol, Dihydrocarveol, Borneol, Perillaldehyde, Perillaketone, oleoylethanolamide, N-oleoyldopamine, 3-methyl-N-oleoyldopamine, oleamide, 1-monoacylglycerol, 2-monoacylglycerol, 1-Arachidonoylglycerol, 2-arachidonoylglycerol, miogadial, miogatrial, polygodial, terpenes with an alpha,beta-unsaturated 1,4-dialdehyde moiety, sanshool, evodiamine, acesulfame-K, cyclamate, CuSO4, ZnSO4, FeSO4, arvanil, N-arachidonoyl-dopamine, flufenamic acid dopamide, dopamine amide of fenamic acid, 4-hydroxynonenal, 1-[2-(1-adamantyl)ethyl]-1-pentyl-3-[3-(4-pyridyl)propyl]urea, Alosetron, Dolasetron, Clozapine, Quetiapine, Mianserin, Piboserod, L-lysine, boldine, oleoyl LPC, N-oleoyldopamine, olvanil, N-palmitoylethanolamide (PEA) and N-oleoylethanolamide (OEA) and combinations thereof.

According to some embodiments, the composition further comprises at least one terpene selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

According to some embodiments, the method further comprises administering to the patient cannabidiol.

According to some embodiments, the at least one modulator and the cannabidiol are administered in separate compositions.

According to some embodiments, the composition comprises between 0.1 and 100 milligram cannabidiol.

### DETAILED DESCRIPTION OF THE INVENTION

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention.

As used herein, the term “anandamide deficiency” refers to an abnormal amount of anandamide levels in the endocannabinoid system and/ or to abnormal functioning of anandamide in the endocannabinoid system. Abnormal amount of anandamide levels in the endocannabinoid system refer to a level of anandamide which is at least 10% less (such as 10% less, 15% less, 20% less, 25% less, 30% less, 35% less, 40% less, 45% less or even 50% less) than the average anandamide level, and/or at least 10% less than the median anandamide level in a healthy subject. Abnormal amount of anandamide levels in the endocannabinoid system refer to a level of anandamide which is at least 10% less (such as 10% less, 15% less, 20% less, 25% less, 30% less, 35% less, 40% less, 45% less or even 50% less) than the average anandamide level, and/or at least 10% less (such as 10% less, 15% less, 20% less, 25% less, 30% less, 35% less, 40% less, 45% less or even 50% less) than the median anandamide level in a healthy subject at the same age and gender. According to some embodiments, anandamide deficiency refers to an abnormal amount and/or functioning of anandmide in the central nervous system, in the peripheral nervous system, in the plasma, in the serum, and/or in the cerebrospinal fluid.

As used herein, the term “modulator” refers to a substance that affects the activity of the endocannabinoid system, e.g. via binding to and/or altering the activity of a receptor. A modulator may include, for example, a ligand, an activator, a full agonist, a partial agonist, an antagonist, an inverse agonist, a positive allosteric modulator and a negative alloseric modulator.

As used herein the term “treating” includes preventing, curing, ameliorating, mitigating, and reducing the instances or severity of a condition or a symptom thereof.

As used herein, the term “CBD” refers to CBDa (cannabidiolic acid) and/or to CBD (cannabidiol) unless indicated otherwise.

Unless indicated otherwise, the term “cannabinoid” as used herein refers to a compound that affects the endocannabinoid system and includes the decarboxylated form of the compound, the acid form of the compound, or combinations thereof. Cannabinoids are agonists or antagonists to receptors in the endocannabinoid system. As used herein, cannabinoid may comprise an endogenous cannabinoid, also known as an endocannabinoid or an exogenous cannabinoid, extracted from a cannabis plant, from a hemp plant or manufactured artificially.

As used herein, the term “THC” refers to THCa (tetrahydrocannabiniolic acid) and/or to THC (tetrahydrocannabiniol) unless indicated otherwise. As used herein, the term “CBG” refers to CBGa (cannabigerolic acid) and/or to CBG (cannabigerol) unless indicated otherwise. As used herein, the term “CBN” refers to CBNa (Cannabinolic acid) and/or to CBN (cannabinol) unless indicated otherwise. As used herein, the term “CBC” refers to CBCa (cannabichromenic acid) and/or to CBC (Cannabichromene) unless indicated otherwise. As used herein, the term “CBL” refers to CBLa (Cannabicycol acid) and/or to CBL (Cannabicyclol) unless indicated otherwise. As used herein, the term “THCV” refers to THCVa (tetrahydrocannabivarin acid) and/or to THCV (tetrahydrocannabivarin) unless indicated otherwise. As used herein, the term “CBDV” refers to CBDVa (cannabigerovarin acid) and/or to CBDV (cannabidivarin) unless indicated otherwise.

As used herein, the term “terpene” refers to both terpenes and terpenoids.

As used herein, the term “2-AG deficiency” refers to an abnormal amount of 2-AG levels in the endocannabinoid system and/or to abnormal functioning of 2-AG in the endocannabinoid system. Abnormal amount of 2-AG levels in the endocannabinoid system refer to a level of 2-AG which is at least 10% less (such as 10% less, 15% less, 20% less, 25% less, 30% less, 35% less, 40% less, 45% less or even 50% less) than the average 2-AG level, and/or at least 10% less (such as 10% less, 15% less, 20% less, 25% less, 30% less, 35% less, 40% less, 45% less or even 50% less) than the median 2-AG level in a healthy subject at the same age and gender. According to some embodiments, 2-AG deficiency refers to an abnormal amount and/or functioning of 2-AG in the central nervous system, in the peripheral nervous system, in the plasma, in the serum, and/or in the cerebrospinal fluid.

Unless indicated otherwise, percent is weight percent and ratio is weight/weight ratio. Unless indicated otherwise, weight ratio means the ratio between weight content.

As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

As used herein, when a numerical value is preceded by the term“about”, the term “about” is intended to indicate ±10% of that value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

According to an aspect of some embodiments of the present invention, there is provided a pharmaceutical composition comprising a carrier and a pharmaceutically effective amount of at least one modulator of an activity which is responsive to modulation by at least one selected from the group consisting of anadamide, N-Oleoylethanolamine (OEA), N-palmitoylethanolamind (PEA) and seretonine and is responsive to modulation by 2-arachidonoylglycerol (2-AG) to an extent that is no greater than that provided by at least one of anadamide, OEA, PEA and seretonine at the same molar dose, wherein the composition is devoid of a sole modulator selected from the group consisting of cannabidiol (CBD), anadamide, N-Oleoylethanolamine (OEA), N-palmitoylethanolamind (PEA) and seretonine, for use in treating a condition characterized by anandamide deficiency or a symptom thereof.

According to one embodiment, the activity which is responsive to modulation by at least one selected from the group consisting of anadamide, N-oleylethanolamine (OEA), N-palmitoylethanolamind (PEA) and seretonine and is responsive to modulation by 2-arachidonoylglycerol (2-AG) to an extent that is no greater than that provided by at least one of anadamide, OEA, PEA and seretonine at the same molar dose, comprises reduction of stress response via inhibition of the HPA axis, responsive to modulation by anadamide. According to another embodiment, said activity comprises analgesia via activation and desensitization the TRPV1 channels responsive to modulation by anadamide. According to another embodiment, said activity comprises analgesia via activation and desensitization the TRPA1 channels responsive to modulation by anadamide. According to another embodiment, said activity comprises reducing hyperanalgesia, leading to increased pain perception, self injury behavior, hyper-sensory processing, hypersensitivity and combination thereof, via activation and desensitization the TRPV1 and or TRPA1 channels responsive to modulation by anadamide. According an embodiment, said activity comprises reducing cold sensation and cold hyperanalgsia via antagonism of TRPM8 responsive to modulation by anadamide. According to another embodiment, said activity comprises reducing impaired thermo-regulation via antagonism of TRPM8 responsive to modulation by anadamide and/or agonism of TRPV1 responsive to modulation by anandamide. According to another embodiment, said activity comprises improving glocuse homeostatis via GRP119 activation by OEA. According to another embodiment, said activity comprises regulating feeding and body weight via activation of receptor peroxisome proliferator-activated receptor (PPAR) responsive to modulation by OEA. According to another embodiment, said activity comprises analgesia and anti-inflammatory activity via mediating PPAR responsive to modulation by PEA. According to another embodiment, said activity comprises reduction of stress via modulating serotonine receptors. According to another embodiment, said activity comprises reduction of stress via antagonising serotonine receptors and increasing anadomide availability. According to another embodiment, said activity comprises reduction of stress via increasing CB1 receptors expression responsive to modulation by anadomide. According to another embodiment, said activity comprises reduction of stress via antagonising serotonine receptors and increasing CB1 receptors expression.

According to some embodiments, said anandmide deficiency to be treated, results from increased Fatty Acid Amide Hydrolase (FAAH) activity, probably leading to increased anandamide hydrolysis. According to other embodiments, said anandmide deficiency to be treated, results from reduced activity of N-acyl-phosphatidylethanolamine-specific phospholipase D(NAPE-PLD) activity, probably leading to reduced anadomide synthesis.

According to some embodiments, said anadmide deficiency further comprises abnormal amount and/or functioning of N-Oleoylethanolamine (OEA), and/or N-palmitoylethanolamind (PEA). Abnormal amount of OEA, and/or PEA levels refer to a level of OEA, and/or PEA which is at least 10% less (such as 10% less, 15% less, 20% less, 25% less, 30% less, 35% less, 40% less, 45% less or even 50% less) than the average OEA and/ or PEA levels, and/or at least 10% less than the median OEA and/or PEA level in a healthy subject.

According to some embodiments, said at least one modulator is selected from the group consisting of transient receptor potential (TRP) channel modulators, peroxisome proliferator-activated receptor (PPAR) modulators, serotonin (5-HT) receptor modulators, glycine receptor (GlyR) modulators, G protein-coupled receptor modulators, and combinations thereof.

According to some embodiments, said at least one modulator is of an activity responsive to modulation by anadamide and is responsive to modulation by 2-arachidonoylglycerol (2-AG) to an extent that is no greater than that provided by anadamide at the same molar dose.

According to some embodiments, said activity is responsive to modulation by at least one selected from the group consisting of anadamide, N-Oleoylethanolamine (OEA), N-palmitoylethanolamind (PEA) and is not responsive to modulation by 2-arachidonoylglycerol (2-AG).

According to some embodiments, said activity is responsive to modulation by at least one selected from the group consisting of anadamide, N-Oleoylethanolamine (OEA), N-palmitoylethanolamind (PEA) and seretonine to an extent that is at least 10% greater than to modulation by 2-arachidonoylglycerol (2-AG), at least 20%, at least 30%, at least 40% or at least 50% greater.

According to some embodiments, said at least one modulator comprises at least one TRP channel modulator. According to some such embodiments, said at least one TRP channel modulator comprises at least one TRPV1 channel modulator, such as at least one, at least two, at least three, at least four or at least five TRP channel modulators.

According to some embodiments, said at least one modulator comprises at least two TRP channel modulators, such as at least two, at least three, at least four or even at least five TRP channel modulators.

According to some embodiments, said G protein-coupled receptor modulators comprises G protein-coupled receptor 119 (GPR119) receptor modulators, cannabinoid receptor type 1 (CB1) modulator or both.

According to an embodiment, said carrier is selected from the group consisting of vegetable oils, e.g. coconut oil, olive oil or sesame oil, pharmaceutical excipients, honey, bees wax, cellulose and combinations thereof. As used herein, the term cellulose refers to cellulose, hemicellulose and their combinations.

According to some embodiments, said at least one modulator further comprises neutral cannabidiol (CBD) and/or cannabiodiolic acid (CBDA). According to some such embodiments, said CBD is present in the composition at less than 50% of a total molar amount of said at least one modulator.

According to some embodiments, said at least one modulator further comprises neutral tetrahydrocannabinol (THC) and/or tetrahydrocannbinolic acid.

According to some embodiments, said at least one modulator further comprises cannabidiol and tetrahydrocannabinol, wherein a cannabidiol to tetrahydrocannabinol weight per weight ratio is greater than 2:1, such as greater than 2:1, at least 3:1, at least 4:1, at least 5:1, at least 6:1, at least 7:1, at least 8:1, at least 9:1 or at least 10:1.

According to some embodiments, the composition further comprises cannabivarin.

According to some embodiments, said at least one modulator comprises at least one selected from the group consisting of N-palmitoylethanolamide (PEA), N-oleoylethanolamide (OEA) and combinations thereof and at least a second modulator.

According to some embodiments, said condition involves no significant 2-AG deficiency.

According to some embodiments, said condition is an autistic spectrum disorder.

According to some embodiments, said condition is selected from the group consisting of migraine, fibromyalgia, irritable bowel diseases, diabetes, post-traumatic stress disorders, chronic stress, anxiety, stress-related psychiatric illness, hyperanalgesia affective disorders and combinations thereof.

According to some embodiments, said symptom is selected from the group consisting of increased pain perception, reduced pain perception, headache, self-injury behavior, hyper-sensory processing, hypo-sensory processing, impaired sensory processing, hypersensitivity, hyposensitivity, impaired thermo-regulation, inflammation, digestive problems, nausea, vomiting, constipation, urinary-system related problems, impaired memory, impaired learning, seizures, anxiety, stress, fear, aversive emotion, aversive emotional memories, mood, depression, dermatitis, impaired social cognition, impaired social behavior, sleep problems, insomnia and combinations thereof.

According to some embodiments, said at least one modulator comprises a cannabinoid.

According to some embodiments, said at least one modulator comprises a terpene.

According to some embodiments, said at least one modulator is selected from the group consisting of Capsaicin, capsaicinoid, capsinoid, Capsiate, Dihydrocapsiate, Nordihydrocapsiate, Piperine, Eugenol, Resiniferatoxin, Gingerol, Shogaol, Zingerone, Paradol, Grifolin, Neogrifolin, Albaconol, Scutigeral, Cochinchinenin, Loureirin, Allyl isothiocyanate, Cinnamaldehyde, Allicin, Diallyl disulphide, Ligustilide, Dehydroligustilide, Alpha-spinasterol, Guaiacol, Methyl salicylate, Vanillin, Methyl syringate, Resveratrol, Pinosylvin methyl ether, Salidroside, Bisandrographolide, Ginsenoside, Triptolide, Evodiamine, Nicotine, Yohimbine, Hydroxy-alphasanshool, Artepillin, Thapsigargin, Ricinoleic acid, Incensole acetate, Hyperforin, Vanillotoxins, Quinine, Waixenicin, Citral, Camphor, Carvacrol, Thymol, Menthol, cineole, Perillaldehyde, Perillaketone, Polygodial, Drimanial, Isovelleral, Cinnamodial, Cinnamosmolide, Cinnamolide, Warburganal, Scalaradial, Aframodial, Ancistrodial, Merulidial, Drimenol, Umbellulone, Cineole, Carveol, 6-tert-butyl-m-cresol Dihydrocarveol, Borneol, Perillaldehyde, Perillaketone, oleoylethanolamide, N-oleoyldopamine, 3-methyl-N-oleoyldopamine, oleamide, 1-monoacylglycerol, 2-monoacylglycerol, 1-Arachidonoylglycerol, 2-arachidonoylglycerol, miogadial, miogatrial, polygodial, terpenes with an alpha,beta-unsaturated 1,4-dialdehyde moiety, sanshool, evodiamine, acesulfame-K, cyclamate, CuSO4, ZnSO4, FeSO4, arvanil, N-arachidonoyl-dopamine, flufenamic acid dopamide, dopamine amide of fenamic acid, 4-hydroxynonenal, 1-[2-(1-adamantyl)ethyl]-1-pentyl-3-[3-(4-pyridyl)propyl]urea, Alosetron, Dolasetron, Clozapine, Quetiapine, Mianserin, Piboserod, L-lysine, blodine, oleoyl LPC, N-oleoyldopamine, olvanilN-palmitoylethanolamide (PEA) and N-oleoylethanolamide (OEA) and combinations thereof.

According to some embodiments, said modulator comprises a CB1 receptor modulator. According to some embodiments, said at least one TRP channel modulator comprises a CB1 receptor modulator. According to some embodiments, said at least one TRP channel modulator comprises a CB1 receptor agonist.

According to some embodiments, the composition further comprises at least one terpene selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

According to an aspect of some embodiments of the present invention, there is provided a pharmaceutical composition comprising a carrier and a pharmaceutically effective amount of at least one modulator selected from the group consisting of transient receptor potential (TRP) channel modulators, peroxisome proliferator-activated receptor (PPAR) modulators, serotonin (5-HT) receptor modulators, glycine receptor (GlyR) receptor modulators, G protein-coupled receptors, and combinations thereof, wherein cannabidiol (CBD) is not present as a sole modulator, for use in treating a condition characterized by anandamide deficiency or a symptom thereof.

According to an embodiment, said at least one modulator comprises at least one TRP channel modulator, such as at least one, at least two, at least three, at least four or at least five TRP channel modulators.

According to an embodiment, said at least one TRP channel modulator comprises at least one TRPV1 channel modulator, such as at least one, at least two, at least three, at least four or at least five TRP channel modulators.

According to an embodiment, said at least one TRP channel modulator comprises at least two TRP channel modulators, such as at least one, at least two, at least three, at least four or at least five TRP channel modulators. According to some such embodiments, one of the at least two TRV1 channel modulators is cannabidiol.

According to an embodiment, said carrier is selected from the group consisting of vegetable oils, e.g. coconut oil, olive oil or sesame oil, pharmaceutical excipients, honey, bees wax, cellulose and combinations thereof. As used herein, the term cellulose refers to cellulose, hemicellulose and their combinations.

According to an embodiment, the pharmaceutical composition comprises said at least one modulator and further comprises cannabidiol.

According to an embodiment, the pharmaceutical composition further comprises tetrahydrocannabinol.

According to an embodiment, the pharmaceutical composition further comprises cannabidiol and tetrahydrocannabinol, wherein a cannabidiol to tetrahydrocannabinol weight per weight ratio is greater than 2:1, such as greater than 2:1, at least 3:1, at least 4:1, at least 5:1, at least 6:1, at least 7:1, at least 8:1, at least 9:1 or at least 10:1.

According to an embodiment, the pharmaceutical composition further comprises cannabivarin.

According to an embodiment, the pharmaceutical composition further comprises at least one selected from the group consisting of N-palmitoylethanolamide (PEA), N-oleoylethanolamide (OEA) and combinations thereof.

According to an embodiment, said condition involves no significant 2-AG deficiency.

According to an embodiment, said condition is an autism spectrum disorder.

According to an embodiment, said condition is selected from the group consisting of migraine, fibromyalgia, irritable bowel diseases, diabetes, post-traumatic stress disorders, affective disorders and combinations thereof.

According to an embodiment, said symptom is selected from the group consisting of increased pain perception, reduced pain perception, headache, self-injury behavior, hyper-sensory processing, hypo-sensory processing, impaired sensory processing, hypersensitivity, hyposensitivity, impaired thermo-regulation, inflammation, digestive problems, nausea, vomiting, constipation, urinary-system related problems, impaired memory, impaired learning, seizures, anxiety, stress, fear, aversive emotion, aversive emotional memories, mood, depression, dermatitis, impaired social cognition, impaired social behavior, sleep problems, insomnia and combinations thereof.

According to an embodiment, said TRP channel modulator comprises a cannabinoid.

According to an embodiment, said at least one TRP channel modulator comprises a terpene.

According to an embodiment, said at least one TRP channel modulator is selected from the group consisting of Capsaicin, capsaicinoid, capsinoid, Capsiate, Dihydrocapsiate, Nordihydrocapsiate, Piperine, Eugenol, Resiniferatoxin, Gingerol, Shogaol, Zingerone, Paradol, Grifolin, Neogrifolin, Albaconol, Scutigeral, Cochinchinenin, Loureirin, Allyl isothiocyanate, Cinnamaldehyde, Allicin, Diallyl disulphide, Ligustilide, Dehydroligustilide, Alpha-spinasterol, Guaiacol, Methyl salicylate, Vanillin, Methyl syringate, Resveratrol, Pinosylvin methyl ether, Salidroside, Bisandrographolide, Ginsenoside, Triptolide, Evodiamine, Nicotine, Yohimbine, Hydroxy-alphasanshool, Artepillin, Thapsigargin, Ricinoleic acid, Incensole acetate, Hyperforin, Vanillotoxins, Quinine, Waixenicin, Citral, Camphor, Carvacrol, Thymol, Menthol, cineole, Perillaldehyde, Perillaketone, Polygodial, Drimanial, Isovelleral, Cinnamodial, Cinnamosmolide, Cinnamolide, Warburganal, Scalaradial, Aframodial, Ancistrodial, Merulidial, Drimenol, Umbellulone, Cineole, Carveol, 6-tert-butyl-m-cresol, Dihydrocarveol, Borneol, Perillaldehyde, Perillaketone, oleoylethanolamide, N-oleoyldopamine, 3-methyl-N-oleoyldopamine, oleamide, 1-monoacylglycerol, 2-monoacylglycerol, 1-Arachidonoylglycerol, 2-arachidonoylglycerol, miogadial, miogatrial, polygodial, terpenes with an alpha,beta-unsaturated 1,4-dialdehyde moiety, sanshool, evodiamine, acesulfame-K, cyclamate, CuSO4, ZnSO4, FeSO4, arvanil, N-arachidonoyl-dopamine, flufenamic acid dopamide, dopamine amide of fenamic acid, 4-hydroxynonenal, 1-[2-(1-adamantyl)ethyl]-1-pentyl-3-[3-(4-pyridyl)propyl]urea, N-palmitoylethanolamide (PEA) and N-oleoylethanolamide (OEA) and combinations thereof.

According to some embodiments, said modulator comprises a CB1 receptor modulator. According to some embodiments, said at least one TRP channel modulator comprises a CB1 receptor modulator. According to an embodiment, said at least one TRP channel modulator comprises a CB1 receptor modulator.

According to an embodiment, the pharmaceutical composition for use further comprises at least one terpene selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene, friedelin, carvacrol, eugenol, camphor, menthol, iso-menthone, neral, gerial, viridiflorol, germacrene, thymol, Menth-2-en-1-ol, farensol, carotol, myrtenol and combinations thereof. According to some such embodiments, the pharmaceutical composition comprises at least one, at least two, at least three, at least four or at least five such terpenes. In some such embodiments, the terpene does not function as a modulator.

According to an embodiment, the pharmaceutical composition for use further comprises an additive selected from the group consisting of antioxidants, emulsifiers and texturizers vegetable oils, plant extracts, honey, pharmaceutical excipients, sucrose, glucose and fructose, pharmaceutical excipients and combinations thereof. According to an embodiment, the composition comprises a surfactant selected from the group consisting of phospholipids, glycerides, glycolipids and combinations thereof. According to an embodiment, the composition further comprises a food-approved texturizer. According to an embodiment, the composition further comprises at least 10 ppm ethanol. According to an embodiment, the composition further comprises at least one of vitamin C, vitamin E, polyunsaturated fatty acids, beeswax and coconut oil. According to an embodiment, the composition further comprises a sweetener.

According to an aspect of an embodiment of the present invention, there is provided a pharmaceutical preparation comprising the pharmaceutical composition for use as disclosed herein.

According to an embodiment, the pharmaceutical preparation comprises between 0.1 and 100 milligram modulator, such as at least 0.1, about 0.2, about 0.3, about 0.4, about 0.5, about 0.6, about 0.7, about 0.8, about 0.9, about 1, about 2, about 3, about 4, about 5, about 6, about 7, about 8, about 9, about 10, about 20, about 30, about 40, about 50, about 60, about 70, about 80, about 90 and up to 100 milligram modulator.

According to an embodiment, the preparation comprises the composition in a dosage form suitable for oral administration, such as, for example, as a pill, tablet, capsule, syrup, solution, suspension, powder or the like, or a combination thereof.

According to an embodiment, the preparation comprises the composition in a form suitable for administration by inhalation, such as, for example, in an aerosol, inhaler, nebulizer, vaporizer or the like, or combinations thereof.

According to an embodiment, the preparation comprises the composition for use in a form suitable for parenteral administration, such as for example, for administration by intradermal, subcutaneous, intramuscular, intraosseous, intraperitioneal or intravenous injection, or any combination thereof.

According to an embodiment, the preparation comprises a suppository, such as a vaginal suppository (including a douche, pessary or the like), a rectal suppository, urethral suppository or nasal suppository, or any combination thereof.

According to an embodiment, the preparation comprises a cigarette.

According to an embodiment, the preparation comprises the composition in a form suitable for topical administration, such as, for example, in a cream, ointment, gel, oil, patch or combinations thereof.

According to an embodiment, the pharmaceutical preparation comprises cannabidiol. In some such embodiments, said preparation comprises between 0.1 and 100 milligram cannabidiol, such as at least 0.1, about 0.2, about 0.3, about 0.4, about 0.5, about 0.6, about 0.7, about 0.8, about 0.9, about 1, about 2, about 3, about 4, about 5, about 6, about 7, about 8, about 9, about 10, about 20, about 30, about 40, about 50, about 60, about 70, about 80, about 90 and up to 100 miligram cannabidiol.

According to an aspect of some embodiments of the present invention, there is provided a kit comprising a first pharmaceutical preparation and a second pharmaceutical preparation, wherein each of said first and said second preparations comprises both cannabidiol and tetrahydrocannabinol and wherein the cannabidiol to tetrahydrocannabinol weight per weight ratio in said first preparation is greater than a cannabidiol to tetrahydrocannabinol weight per weight ratio in said second preparation. According to some embodiments of the present invention, said cannabidiol to tetrahydrocannabinol weight per weight ratio in both preparations is greater than 2:1, such as greater than 2:1, at least 3:1, at least 4:1, at least 5:1, at least 6:1, at least 7:1, at least 8:1, at least 9:1 or at least 10:1.

According to an aspect of some embodiments of the present invention, there is provided a kit comprising a first pharmaceutical preparation and a second pharmaceutical preparation, wherein each of said first and said second preparations comprises at least one terpene, and wherein the terpene content of said first preparation differs from the terpene content of said second preparation. In some such embodiments, the terpene content of the first preparation differs from that of the second preparation in that different terpenes are present. Additionally, or alternatively, the terpene content of the first preparation differs from that of the second preparation in that different concentrations and/or ratios of terpenes are present.

According to an aspect of some embodiments of the present invention, there is provided a method for treating a condition characterized by anandamide deficiency or a symptom thereof in a subject in need thereof, comprising administering to the subject a pharmaceutical composition comprising a carrier and a pharmaceutically effective amount of at least one modulator of an activity responsive to modulation by at least one selected from the group consisting of anadamide, N-Oleoylethanolamine (OEA), N-palmitoylethanolamind (PEA) and seretonine and modulated by 2-arachidonoylglycerol (2-AG) to an extent that is no greater than that provided by at least one of anadamide, OEA, PEA and seretonine at the same molar dose, wherein the composition is devoid of a sole modulator selected from the group consisting of cannabidiol (CBD), anadamide N-Oleoylethanolamine (OEA), N-palmitoylethanolamind (PEA) and seretonine.

According to an aspect of some embodiments of the present invention, there is provided a method for treating a condition characterized by anandamide deficiency or a symptom thereof in a subject in need, comprising administering to the subject a preparation comprising a composition comprising a carrier and a pharmaceutically effective amount of at least one modulator of an activity responsive to modulation by at least one selected from the group consisting of anadamide, N-Oleoylethanolamine (OEA), N-palmitoylethanolamind (PEA) and seretonine and modulated by 2-arachidonoylglycerol (2-AG) to an extent that is no greater than that provided by at least one of anadamide, OEA, PEA and seretonine at the same molar dose, wherein the composition is devoid of a sole modulator selected from the group consisting of cannabidiol (CBD), anadamide N-Oleoylethanolamine (OEA), N-palmitoylethanolamind (PEA) and seretonine.

According to an aspect of some embodiments of the present invention, there is provided a method for treating a condition characterized by anandamide deficiency or a symptom thereof in a subject in need, comprising administering to the subject a pharmaceutical composition comprising a carrier and a pharmaceutically effective amount of at least one modulator selected from the group consisting of transient receptor potential (TRP) channel modulators, peroxisome proliferator-activated receptor (PPAR) modulators, serotonin (5-HT) receptor modulators, glycine receptor (GlyR) receptor modulators, G protein-coupled receptors, and combinations thereof, wherein cannabidiol (CBD) is not present as a sole modulator.

According to some embodiments of the method, said G protein-coupled receptor modulators comprises G protein-coupled receptor 119 (GPR119) receptor modulators, cannabinoid receptor type 1 (CB1) modulator or both.

According to some embodiments of the method, said at least one modulator further comprising neutral cannabidiol (CBD) and/or cannabiodiolic acid (CBDA). According to some such embodiments, said CBD is present in the composition at less than 50% of a total molar amount of said at least one modulator.

According to an aspect of some embodiments of the present invention, there is provided a method for treating a condition characterized by anandamide deficiency or a symptom thereof in a subject in need, comprising administering to the subject a preparation comprising a pharmaceutical composition comprising a carrier and a pharmaceutically effective amount of at least one modulator selected from the group consisting of transient receptor potential (TRP) channel modulators, peroxisome proliferator-activated receptor (PPAR) modulators, serotonin (5-HT) receptor modulators, glycine receptor (GlyR) receptor modulators, G protein-coupled receptors, and combinations thereof, wherein cannabidiol (CBD) is not present as a sole modulator.

According to an aspect of some embodiments of the present invention, there is provided the use of a pharmaceutical composition comprising a carrier and a pharmaceutically effective amount of at least one modulator selected from the group consisting of transient receptor potential (TRP) channel modulators, peroxisome proliferator-activated receptor (PPAR) modulators, serotonin (5-HT) receptor modulators, glycine receptor (GlyR) receptor modulators, GPR119 receptor modulators, and combinations thereof, wherein CBD is not present as a sole modulator, in the manufacture of a medicament for the treatment of a condition characterized by anandamide deficiency or a symptom thereof.

According to some embodiments of the method, said G protein-coupled receptor modulators comprises G protein-coupled receptor 119 (GPR119) receptor modulators, cannabinoid receptor type 1 (CB1) modulator or both.

According to some embodiments of the method, said at least one modulator further comprising neutral cannabidiol (CBD) and/or cannabiodiolic acid (CBDA). According to some such embodiments, said CBD is present in the composition at less than 50% of a total molar amount of said at least one modulator.

According to an embodiment, said condition involves no significant 2-AG deficiency.

According to an embodiment, said condition is selected from the group consisting of autistic spectrum disorders, migraine, fibromyalgia, irritable bowel diseases, diabetes, post-traumatic stress disorders, affective disorders and combinations thereof.

According to an embodiment, said symptom is selected from the group consisting of increased pain perception, reduced pain perception, headache, self-injury behavior, hyper-sensory processing, hypo-sensory processing, impaired sensory processing, hypersensitivity, hyposensitivity, impaired thermo-regulation, inflammation, digestive problems, nausea, vomiting, constipation, urinary-system related problems, impaired memory, impaired learning, seizures, anxiety, stress, fear, aversive emotion, aversive emotional memories, mood, depression, dermatitis, impaired social cognition, impaired social behavior, sleep problems, insomnia and combinations thereof.

According to an embodiment, the method further comprises administering to said patient cannabidiol.

According to an embodiment, said at least one modulator and said cannabidiol are administered in separate compositions. In some such embodiments, said separate compositions comprise different dosage forms. In some such embodiments, said separate compositions comprise same dosage forms, wherein said dosage form comprising said modulator is separate from said dosage form comprising said cannabidiol.

According to an embodiment, the at least one modulator and the cannabidiol are administered in separate dosage forms independently, sequentially, simultaneously or concomitantly in separate compositions. In some embodiments, wherein the at least one modulator and the cannabidiol are administered sequentially, the at least one modulator is administered first, followed by the cannabidiol. In some embodiments, wherein the at least one modulator and the cannabidiol are administered sequentially, the cannabidiol is administered first, followed by the at least one modulator.

According to an embodiment, the condition is an autism spectrum disorder.

It is intended that the specification and examples be considered as exemplary only, and that the scope of the invention shall include all modifications and variations that may be apparent to those skilled in the art from consideration of the specification and practice of the invention as disclosed herein.

### EXAMPLES

| | Dose | | | Content | | Therapeutic effect |
|---|---|---|---|---|---|---|
| Example | (mg) | Form | Modulator | (mg) | Carrier | For: |
| 1 | 150 | Sub-liguial | CBD | 10 | Olive oil | Stress, anxiety |
| | | oil | Vanillin | 6 | | |
| 2 | 500 | Topical | Camphor | 20 | Wax | Imparied |
| | | | Menthol | 50 | | termoregulation |
| 3 | 200 | Sub- | Myrcene | 4 | MCT | Hyperalgesia |
| | | lignual oil | | | | |
| 4 | 250 | Tablet | Myrcene | 10 | Glucose | Inflammation |
| | | | Nerolidol | 10 | | |
| 5 | 400 | Tablet | CBD | 20 | Glucose | Digestive problems |
| | | | Limonene | 8 | | |
| | | | Olvanil | 5 | | |
| 6 | 90 | Sub-ligual | Citral | 5 | Olive oil | Hypersensitivity |
| | | oil | Geraniol | 5 | | |
| | | | Citronellol | 5 | | |
| 7 | 300 | Tablet | CBD | 10 | Glucose | Anxiety, stress-related |
| | | | Menthol | 15 | | psychiatric illness |
| 8 | 200 | Gel | Boldine | 3 | MCT | Stress |
| | | capsules | Geraniol | 4 | | |
| | | | Linalool | 6 | | |
| | | | Gingerol | 4 | | |
| 9 | 500 | Tablet | Caryophyllene | 10 | Cellulose | feeding and body weight |
| | | | CBD | 10 | | regulation |
| 10 | 120 | Sub-ligual | Caryophyllene | 8 | Olive oil | Digestive problems |
| | | oil | Eucalyptol | 4 | | |
| | | | Geraniol | 4 | | |
| 11 | 1000 | Topical | Eugenol | 20 | Wax | Hyposensitivity |
| | | | Cinnamodial | 20 | | |
| | | | Linalool | 20 | | |
| 12 | 500 | Topical | Borneol | 20 | Wax | migraine |
| | | | Camphor | 10 | | |
| | | | Eugenol | 10 | | |
| 13 | 90 | Sub-lingual | CBD | 10 | MCT | Anxiety |
| | | oil | Linalool | 20 | | |
| 14 | 500 | Topical | Capsaicin | 10 | Lecithin | Hypersensitivity, self |
| | | | Zingerone | 10 | Wax | injury |
| | | | Gingerol | 30 | | |
| 15 | 250 | Tablet | N-oleoyldopa- | 3 | Glucose | Improving food intake |
| | | | mine | | | |
| | | | Olvanil | 3 | | |
| 16 | 200 | Topical | CBD | 5 | Lecithin | increased pain |
| | | | Capsaicin | 20 | Wax | perception |
| 17 | 120 | Sub-linguial | CBD | 12 | MCT | Fibromyalgia |
| | | drops | Citronellol | 5 | | |
| | | | Menthol | 8 | | |
| 18 | 500 | Tablet | oleoyl LPC | 5 | Cellulose | Diabetes |
| | | | N-oleoyldopan | 5 | | |
| | | | Caryophyllene | 10 | | |
| | | | CBD | 10 | | |
| 19 | 400 | Tablet | CBD | 8 | Glucose | irritable bowel diseases |
| | | | Gingerol | 8 | | |
| | | | Eugenol | 8 | | |
| 20 | 60 | Sub-lingual | 1,8-cineole | 5 | Olive oil | Inflammation |
| | | oil | CBD | 8 | | |
| | | | Pinene | 5 | | |
| 21 | 500 | Tablet | CBD | 20 | Cellulose | impaired social behavior |
| | | | Linalool | 5 | | |
| | | | Menthol | 20 | | |
| 22 | 500 | Topical | CBD | 25 | Lecithin | Increased pain perception |
| | | | Piperine | 10 | Wax | |
| | | | Linalool | 50 | | |

What is claimed is:

1. A pharmaceutical composition comprising a carrier and a pharmaceutically effective amount of at least two modulators of an activity which activity is responsive to modulation by at least one selected from the group consisting of anadamide, N-Oleoylethanolamine (OEA), N-palmitoylethanolamind (PEA) and seretonine and which activity is responsive to modulation by 2-arachidonoylglycerol (2-AG) to an extent that is no greater than that provided by at least one of anadamide, OEA, PEA and seretonine at the same molar dose.

2. The pharmaceutical composition according to claim 1, wherein at least one modulator is selected from the group consisting of transient receptor potential (TRP) channel modulators, TRPV1 channel modulators, peroxisome proliferator-activated receptor (PPAR) modulators, serotonin (5-HT) receptor modulators, glycine receptor (GlyR) modulators, G protein-coupled receptor modulators, G protein-coupled receptor 119 (GPR119) receptor modulators, cannabinoid receptor type 1 (CB1) modulators and combinations thereof.

3. The pharmaceutical composition according to claim 1, wherein at least one modulator comprises at least one selected from a group comprising neutral cannabidiol (CBD), cannabiodiolic acid (CBDA) neutral tetrahydrocannabinol (THC), tetrahydrocannbinolic acid (THCA), cannabivarin (CBDV), of N-palmitoylethanolamide (PEA), N-oleoylethanolamide (OEA), terpenes and combination thereof.

4. The pharmaceutical composition according to claim 1, wherein at least one modulator is selected from the group consisting of Capsaicin, capsaicinoid, capsinoid, Capsiate, Dihydrocapsiate, Nordihydrocapsiate, Piperine, Eugenol, Resiniferatoxin, Gingerol, Shogaol, Zingerone, Paradol, Grifolin, Neogrifolin, Albaconol, Scutigeral, Cochinchinenin, Loureirin, Allyl isothiocyanate, Cinnamaldehyde, Allicin, Diallyl disulphide, Ligustilide, Dehydroligustilide, Alpha-spinasterol, Guaiacol, Methyl salicylate, Vanillin, Methyl syringate, Resveratrol, Pinosylvin methyl ether, Salidroside, Bisandrographolide, Ginsenoside, Triptolide, Evodiamine, Nicotine, Yohimbine, Hydroxy-alphasanshool, Artepillin, Thapsigargin, Ricinoleic acid, Incensole acetate, Hyperforin, Vanillotoxins, Quinine, Waixenicin, Citral, Camphor, Carvacrol, Thymol, Menthol, cineole, Perillaldehyde, Perillaketone, Polygodial, Drimanial, Isovelleral, Cinnamodial, Cinnamosmolide, Cinnamolide, Warburganal, Scalaradial, Aframodial, Ancistrodial, Merulidial, Drimenol, Umbellulone, Cineole, Carveol, 6-tert-butyl-m-cresol, Dihydrocarveol, Borneol, Perillaldehyde, Perillaketone, oleoylethanolamide, N-oleoyldopamine, 3-methyl-N-oleoyldopamine, oleamide, 1-monoacylglycerol, 2-monoacylglycerol, 1-Arachidonoylglycerol, 2-arachidonoylglycerol, miogadial, miogatrial, polygodial, terpenes with an alpha,beta-unsaturated 1,4-dialdehyde moiety, sanshool, evodiamine, acesulfame-K, cyclamate, CuSO4, ZnSO4, FeSO4, arvanil, N-arachidonoyl-dopamine, flufenamic acid dopamide, dopamine amide of fenamic acid, 4-hydroxynonenal, 1-[2-(1-adamantyl)ethyl]-1-pentyl-3-[3-(4-pyridyl)propyl]urea, Alosetron, Dolasetron, Clozapine, Quetiapine, Mianserin, Piboserod, L-lysine, boldine, oleoyl LPC, N-oleoyldopamine, olvanil, N-palmitoylethanolamide (PEA) and N-oleoylethanolamide (OEA) and combinations thereof.

4. The pharmaceutical composition according to claim 1, further comprising at least one terpene selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

5. A pharmaceutical preparation comprising the pharmaceutical composition of claim 1.

6. The pharmaceutical preparation of claim 6, wherein said preparation comprises between 0.1 and 100 milligram cannabidiol.

7. A kit comprising a first and a second preparation each comprising the composition according to claim 1, wherein each of said first and said second preparations further comprises both cannabidiol and tetrahydrocannabinol and wherein a cannabidiol to tetrahydrocannabinol weight per weight ratio in said first preparation is greater than a cannabidiol to tetrahydrocannabinol weight per weight ratio in said second preparation.

8. A kit comprising a first and a second preparation each comprising the composition according to claim 1, wherein each of said first and said second preparations comprises at least one terpene, and wherein the terpene content of said first preparation differs from the terpene content of said second preparation.

9. A method for treating a condition characterized by anandamide deficiency or a symptom thereof in a subject in need thereof, comprising administering to the subject a pharmaceutical composition comprising a carrier and a pharmaceutically effective amount of at least one modulator of an activity which activity is responsive to modulation by at least one selected from the group consisting of anadamide, N-Oleoylethanolamine (OEA), N-palmitoylethanolamind (PEA) and seretonine and which activity is responsive to modulation by 2-arachidonoylglycerol (2-AG) to an extent that is no greater than that provided by at least one of anadamide, OEA, PEA and seretonine at the same molar dose, wherein the composition is devoid of a sole modulator selected from the group consisting of cannabidiol (CBD), anadamide N-Oleoylethanolamine (OEA), N-palmitoylethanolamind (PEA) and seretonine.

10. The method of claim 10, wherein said at least one modulator is selected from the group consisting of transient receptor potential (TRP) channel modulators, peroxisome proliferator-activated receptor (PPAR) modulators, serotonin (5-HT) receptor modulators, glycine receptor (GlyR) modulators, G protein-coupled receptors modulators, G protein-coupled receptor 119 (GPR119) receptor modulators, cannabinoid receptor type 1 (CB1) modulators and combinations thereof.

11. The method of claim 10, wherein said at least one modulator further comprises at least one selected from the group consisting of neutral cannabidiol (CBD), cannabiodiolic acid (CBDA), neutral tetrahydrocannabinol (THC), tetrahydrocannbinolic acid (THCA), cannabivarin (CBDV), of N-palmitoylethanolamide (PEA), N-oleoylethanolamide (OEA), terpenes and combination thereof.

12. The method of claim 15, wherein said CBD is present in the composition at less than 50% of a total molar amount of said at least one modulator.

13. The method of claim 10, wherein said condition involves no significant 2-AG deficiency.

14. The method of claim 10, wherein said condition is selected from the group consisting of autistic spectrum disorders, migraine, fibromyalgia, irritable bowel diseases, diabetes, post-traumatic stress disorders, chronic stress, anxiety, stress-related psychiatric illness, hyperanalgesia, affective disorders, increased pain perception, reduced pain perception, headache, self-injury behavior, hyper-sensory processing, hypo-sensory processing, impaired sensory processing, hypersensitivity, hyposensitivity, impaired thermo-regulation, inflammation, digestive problems, nausea, vomiting, constipation, urinary-system related problems, impaired memory, impaired learning, seizures, anxiety, stress, fear, aversive emotion, aversive emotional memories, mood, depression, dermatitis, impaired social cognition, impaired social behavior, sleep problems, insomnia, an autism spectrum disorder and combinations thereof.

15. The method of claim 10, wherein at least one modulator is selected from the group consisting of Capsaicin, capsaicinoid, capsinoid, Capsiate, Dihydrocapsiate, Nordihydrocapsiate, Piperine, Eugenol, Resiniferatoxin, Gingerol, Shogaol, Zingerone, Paradol, Grifolin, Neogrifolin, Albaconol, Scutigeral, Cochinchinenin, Loureirin, Allyl isothiocyanate, Cinnamaldehyde, Allicin, Diallyl disulphide, Ligustilide, Dehydroligustilide, Alpha-spinasterol, Guaiacol, Methyl salicylate, Vanillin, Methyl syringate, Resveratrol, Pinosylvin methyl ether, Salidroside, Bisandrographolide, Ginsenoside, Triptolide, Evodiamine, Nicotine, Yohimbine, Hydroxy-alphasanshool, Artepillin, Thapsigargin, Ricinoleic acid, Incensole acetate, Hyperforin, Vanillotoxins, Quinine, Waixenicin, Citral, Camphor, Carvacrol, Thymol, Menthol, cineole, Perillaldehyde, Perillaketone, Polygodial, Drimanial, Isovelleral, Cinnamodial, Cinnamosmolide, Cinnamolide, Warburganal, Scalaradial, Aframodial, Ancistrodial, Merulidial, Drimenol, Umbellulone, Cineole, Carveol, 6-tert-butyl-m-cresol, Dihydrocarveol, Borneol, Perillaldehyde, Perillaketone, oleoylethanolamide, N-oleoyldopamine, 3-methyl-N-oleoyldopamine, oleamide, 1-monoacylglycerol, 2-monoacylglycerol, 1-Arachidonoylglycerol, 2-arachidonoylglycerol, miogadial, miogatrial, polygodial, terpenes with an alpha,beta-unsaturated 1,4-dialdehyde moiety, sanshool, evodiamine, acesulfame-K, cyclamate, CuSO4, ZnSO4, FeSO4, arvanil, N-arachidonoyl-dopamine, flufenamic acid dopamide, dopamine amide of fenamic acid, 4-hydroxynonenal, 1-[2-(1-adamantyl)ethyl]-1-pentyl-3-[3-(4-pyridyl)propyl]urea, Alosetron, Dolasetron, Clozapine, Quetiapine, Mianserin, Piboserod, L-lysine, boldine, oleoyl LPC, N-oleoyldopamine, olvanil, N-palmitoylethanolamide (PEA) and N-oleoylethanolamide (OEA) and combinations thereof.

16. The method of claim 10, said composition further comprising at least one terpene selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

17. The method of claim 10, further comprising administering to said patient cannabidiol.

18. The method of claim 19, wherein said at least one modulator and said cannabidiol are administered in separate compositions.

19. The method of claim 10, wherein said composition comprises between 0.1 and 100 milligram cannabidiol.
